(19) United States
(12) Patent Application Publication
(10) Pub. No.: US 2010/0317922 A1
(43) Pub. Date: Dec. 16, 2010

(54) ENDOSCOPE SYSTEM

(75) Inventors: Katsunori Kumai, Tokyo (JP); Yoshiaki Sueoka, Tokyo (JP)

Correspondence Address: VOLPE AND KOENIG, P.C., UNITED PLAZA, 30 SOUTH 17TH STREET, PHILADELPHIA, PA, 19103, US

(73) Assignee: OLYMPUS CORPORATION, Tokyo (JP)

(21) Appl. No.: 12/821,987

(22) Filed: Jun. 23, 2010

Related U.S. Application Data
(63) Continuation of application No. PCT/JP2008/072324, filed on Dec. 9, 2008.

(30) Foreign Application Priority Data
Dec. 28, 2007 (JP) 2007-340179

(51) Int. Cl. A61B 1/12

(52) U.S. Cl. 600/118; 600/156

(57) ABSTRACT

An object is to provide an endoscope system that is capable of cooling photoelectric conversion devices provided in an inserted portion while suppressing an increase in an outer diameter of the inserted portion. An endoscope system employed includes a long, thin inserted portion; photoelectric conversion devices that are mounted at a distal end of the inserted portion; a fluid-feed channel that is provided in the inserted portion and that has a fluid-feed port which opens at the distal end of the inserted portion; a radiator that is connected to an intermediate position of the fluid-feed channel and that is provided in a manner enabling heat exchange with the photoelectric conversion devices; and a fluid-supply-direction switching portion that switches a supply direction of cooling fluid fed by the fluid-feed channel to the fluid-feed port side or to the radiator side.

### CROSS REFERENCE TO RELATED APPLICATIONS

This is a continuation of International Application PCT/JP/2008/072324, with an international filing date of Dec. 9, 2008, which is hereby incorporated by reference herein in its entirety. This application claims the benefit of Japanese Patent Application No. 2007-340179, filed on Dec. 28, 2007, the content of which is incorporated herein by reference.

### TECHNICAL FIELD

The present invention relates to an endoscope system that cools photoelectric conversion devices provided at a distal end of an inserted portion.

### BACKGROUND ART

In recent years, an endoscope system has been proposed wherein a photoelectric conversion device such as a light-emitting diode serving as a light source, a CCD serving as an image acquisition device, etc., is built into the distal end of an inserted portion, in order to simplify the apparatus.

Here, the light-emitting diode has the property that the brightness and life-time deteriorate with increasing temperature caused by heat generated when emitting light. Because the inside of the inserted portion is narrow, making it difficult to dissipate heat, there is a problem in that continuous lighting of the light-emitting diode in such an environment considerably reduces the life-time of the light-emitting diode and also reduces the brightness, thereby hindering the procedure of internal observation of a human body or the like.

To cope with the above-described problems, known cooling techniques in the related art(for example, Patent Citations 1 and 2) involve cooling a photoelectric conversion device, such as a light-emitting diode, a CCD, etc., by circulating cooling air, fluid, etc. in the inserted portion.

[Patent Citation 1] Japanese Unexamined Patent Application, Publication No. Hei 5-111453.

[Patent Citation 2] Japanese Unexamined Patent Application, Publication No. Hei 7-227394.

### DISCLOSURE OF INVENTION

In the above-described techniques, however, because, it is necessary to provide a channel for circulating cooling air, fluid, etc., in the inserted portion, the outer diameter of the inserted portion becomes large. Therefore, there is a problem in that, when applied to medical uses, inserting the inserted portion into the human body increases the burden on a patient.

The present invention has been conceived in light of the above-described circumstances, and an object thereof is to provide an endoscope system that is capable of cooling a photoelectric conversion device provided in an inserted portion while suppressing an increase in the outer diameter of the inserted portion.

In order to achieve the above-described object, the present invention employs the following solutions.

A first aspect of the present invention is an endoscope system that has a long, thin inserted portion; a photoelectric conversion device that is mounted at a distal end of the inserted portion; a fluid-feed channel that is provided in the inserted portion and that has a fluid-feed port which opens at the distal end of the inserted portion; a radiator that is connected to an intermediate position of the fluid-feed channel and that is provided in a manner enabling heat exchange with the photoelectric conversion device; and a fluid-supply-direction switching portion that switches a supply direction of cooling fluid fed by the fluid-feed channel to the fluid-feed port side or to the radiator side.

With the first aspect of the present invention, by providing cooling fluid via the fluid-feed channel in a state in which the inserted portion is inserted inside the body, etc., a site facing the fluid-feed port or an observation window for the photoelectric conversion device can be washed. On the other hand, by switching the fluid-supply direction with the fluid-supply switching portion, the cooling fluid is supplied to the radiator side, and the photoelectric conversion device mounted in the distal end of the inserted portion can be cooled. Accordingly, the photoelectric conversion device can be cooled using the fluid-feed channel that feeds the cooling fluid toward the fluid-feed port, and because a separate fluid-feed channel for cooling the photoelectric conversion device need not be provided, it is possible to prevent an increase in the outer diameter of the inserted portion. Here, the photoelectric conversion device is, for example, a light emitting diode or a CCD.

In the above-described first aspect, the fluid-supply-direction switching portion may switch the supply direction using pressure in the fluid-feed channel.

In this way, the fluid-supply direction switching portion is operated by changing the pressure in the fluid-feed channel, and thus, it is possible to feed fluid by selecting the fluid-feed port side or the radiator side. Accordingly, the fluid-supply direction switching portion can be of a simple configuration in which, for example, a spring, etc. is used, instead of a configuration with a solenoid valve, etc. which requires electric power. In addition, because a power cable for operating a solenoid valve, etc. need not be provided in the inserted portion, it is possible to prevent an increase in the outer diameter of the inserted portion.

In the above-described first aspect, when the pressure in the fluid-feed channel is less than a predetermined value, the fluid-supply direction switching portion may switch the supply direction of the cooling fluid to the radiator side and, when the pressure in the fluid-feed channel is at or above the predetermined value, may switch the supply direction of the cooling fluid to the fluid-feed port side.

In this way, it is possible to improve the washability of the site facing the fluid-feed port by setting the cooling fluid supplied to the fluid-feed port side to a high pressure and a high flow rate, and to reduce fluid pressure exerted on the radiator by setting the cooling fluid supplied to the radiator side to a low pressure and a low flow rate.

The above-described first aspect may additionally include a low-heat-generation-mode setting portion that sets the photoelectric conversion device to low-heat-generation modes, when the fluid-supply-direction switching portion switches the supply direction to the fluid-feed port side.

In this way, when the fluid-supply direction switching portion sets the supply direction of the cooling fluid to the fluid-feed side, that is, when cooling of the photoelectric conversion device is not being carried out, the photoelectric conversion device is set to the low-heat-generation modes by the low-heat-generation mode setting portion, and thus, the amount of heat generated can be reduced. Here, for example, a light-emitting diode, a CCD, and the like are used as the photoelectric conversion device, and examples of low-heat-generation modes for these include lowering the light emission level of the light-emitting diode and lowering the operating clock speed of the CCD.

The above-described first aspect may additionally include a suction channel that is provided in the inserted portion, that has a suction port provided at the distal end of the inserted portion, and that sucks liquid or gas from the suction port.

In this way, it is possible to suck liquid or air near the distal end of the inserted portion from the suction port using the suction channel.

The above-described first aspect, in which the fluid-feed channel and the suction channel are connected via the radiator, may additionally include a suction-direction switching portion that switches the suction direction of the suction channel to the suction port side or to the radiator side, wherein the suction-direction switching portion switches the suction direction of the suction channel to the radiator side when the fluid-feeding direction of the fluid-feed channel is set to the radiator side.

In this way, it is possible to suck, with the suction channel, fluid fed for cooling or washing the site facing the fluid-feed port and fluid fed for cooling the radiator, by switching the suction direction of the suction channel with the suction-direction switching portion. Therefore, a return channel for returning fluid used for cooling the radiator need not be separately provided, and thus, it is possible to reduce the outer diameter of the inserted portion.

In the above-described first aspect, the suction-direction switching portion may switch the suction direction using pressure in the suction channel.

In this way, the suction-direction switching portion is actuated by changing the pressure in the suction channel, and thus, it is possible to suck by selecting the suction port side or the radiator side. Accordingly, the suction-direction switching portion can be of a simple configuration in which, for example, a spring, etc. is used, instead of a configuration with a solenoid valve, etc., which requires electric power. In addition, because a power cable for actuating a solenoid valve, etc. need not be provided in the inserted portion, it is possible to prevent an increase in the outer diameter of the inserted portion.

A second aspect of the present invention is an endoscope system that has a long, thin inserted portion; a photoelectric conversion device that is mounted at a distal end of the inserted portion; a fluid-feed channel that is provided in the inserted portion, that has a fluid-feed port which opens at the distal end of the inserted portion, and that feeds cooling fluid to the fluid-feed port; and a radiator that is disposed adjacent to the photoelectric conversion device, that opens to an outer surface of the inserted portion at the distal end thereof, and from which the cooling fluid fed from the fluid-feed channel in communication therewith seeps out. Here, the radiator is constituted of, for example, a porous material having numerous pores.

In the second aspect of the present invention, the cooling fluid in the fluid-feed channel seeps out to the surface of the radiator through the numerous pores, and because the seeped out fluid takes away heat of vaporization from the radiator when vaporizing, heat dissipation can be carried out. Note that, as the porous material constituting the radiator, for example a sintered metal is suitable. In the above-described second aspect, the radiator may have a hydrophilic layer on a heat dissipation surface thereof.

In this way, it is possible to spread the cooling fluid in the fluid-feed channel in the hydrophilic layer and to efficiently carry out heat dissipation from the radiator. Note that, as the hydrophilic layer, for example, oxidized titanium is suitable.

The above-described second aspect may additionally include a temperature detector that detects the temperature of the radiator; and a fluid-feeding level adjusting portion that adjusts a fluid-feeding level to the radiator in accordance with the temperature detected by the temperature detector.

In this way, it is possible to prevent overheating of the radiator by performing temperature management of the radiator with the temperature detector.

The above-described second aspect may additionally include an air-feed channel that is provided in the inserted portion and that opens near the radiator; and an air-feeding portion that feeds air to the air-feed channel.

In this way, it is possible to facilitate vaporization at the radiator by feeding air to the vicinity of the radiator via the air-feed channel with the air-feeding portion.

The above-described second aspect may additionally include a humidity detector that detects the humidity around the radiator, wherein the air-feeding portion feeds air when the humidity detected by the humidity detector is at or above a predetermined value.

In this way, humidity management around the radiator is performed with the humidity detector, and air is fed to the vicinity of the radiator by the air-feeding portion when the detected humidity is at or above the predetermined value, and thus, vaporization at the radiator can be efficiently carried out.

The above-described second aspect may additionally include a suction channel that is provided in the inserted portion, that has a suction port which opens at the distal end of the inserted portion, and that sucks liquid or gas near the suction port; a pressure detector that detects the pressure around the suction port; and a suction level adjusting portion that adjusts a suction level from the suction port in accordance with the pressure detected by the pressure detector.

In this way, it is possible to suck, with the suction channel, the cooling fluid that is heated upon being used for cooling the radiator. In addition, it is possible to set the pressure around the suction port at an appropriate value by performing pressure management around the suction port with the pressure detector.

With the present invention, an advantage is afforded in that photoelectric conversion devices provided in an inserted portion can be cooled while suppressing an increase in the outer diameter of the inserted portion.

### BRIEF DESCRIPTION OF DRAWINGS

FIG. 1 is a schematic diagram for explaining the configuration of an endoscope system according to a first embodiment of the present invention.

FIG. 2 is a schematic diagram for explaining the operation of the endoscope system of FIG. 1 when feeding fluid.

FIG. 3 is a schematic diagram for explaining the operation of the endoscope system of FIG. 1 during suction.

FIG. 4 is a partial enlarged view of the endoscope system according to a first modification of FIG. 1

FIG. 5 is a partial enlarged view for explaining the operation of the endoscope system of FIG. 4 when feeding fluid.

FIG. 6 is a partial enlarged view of the endoscope system according to a second modification of FIG. 1.

FIG. 7 is a partial enlarged view for explaining the operation of the endoscope system of FIG. 6 during suction.

FIG. 8 is a diagram for explaining the operation of the endoscope system according to a third modification of FIG. 1.

FIG. 9 is a partial enlarged view of the endoscope system according to a fourth modification of FIG. 1.

FIG. 10 is a partial enlarged view for explaining the operation of the endoscope system of FIG. 9 when feeding fluid.

FIG. 11 is a schematic diagram for explaining the configuration of the endoscope system according to a fifth modification of FIG. 1.

FIG. 12A is a perspective view for explaining the configuration of an endoscope system according to a second embodiment of the present invention.

FIG. 12B is a sectional view for explaining the configuration of the endoscope system according to the second embodiment of the present invention.

FIG. 13A is a perspective view for explaining the configuration of the endoscope system according to a modification of FIG. 12A.

FIG. 13B is a sectional view for explaining the configuration of the endoscope system according to a modification of FIG. 12B.

FIG. 14A is a perspective view for explaining the configuration of an endoscope system according to a third embodiment of the present invention.

FIG. 14B is a sectional view for explaining the configuration of the endoscope system according to the third embodiment of the present invention.

FIG. 15A is a temperature monitoring flowchart showing internal processing of the endoscope systems of FIGS. 14A and 14B.

FIG. 15B is a humidity monitoring flowchart showing internal processing of the endoscope systems of FIGS. 14A and 14B.

FIG. 15C is a pressure monitoring flowchart showing internal processing of the endoscope systems of FIGS. 14A and 14B.

FIG. 16A is a perspective view for explaining the configuration of the endoscope system according to a modification of FIG. 14A.

FIG. 16B is a sectional view for explaining the configuration of the endoscope system according to a modification of FIG. 14B.

### EXPLANATION OF REFERENCE SIGNS

A: observation site
1, 2, 3: endoscope system
10, 50: inserted portion
11, 52: photoelectric conversion device
12, 51: fluid-feed port
13, 55: fluid-feed channel
14, 58: radiator
15, 31: fluid-supply-direction switching valve
20: operating portion
22, 66: suction port
23, 56: suction channel
25, 35: suction-direction switching valve
41: return channel
61: temperature detector
63: humidity detector
63: pressure detector
68: hydrophilic layer
70: endoscope control unit
71: controller
72: fluid-feeding pump
73: dryer
74: air-feeding pump
75: air filter
76: discharge/exhaust pump
77: tank
91: fluid-feed-suction port

### BEST MODE FOR CARRYING OUT THE INVENTION

### First Embodiment

An endoscope system according to a first embodiment of the present invention will be described below with reference to the drawings.

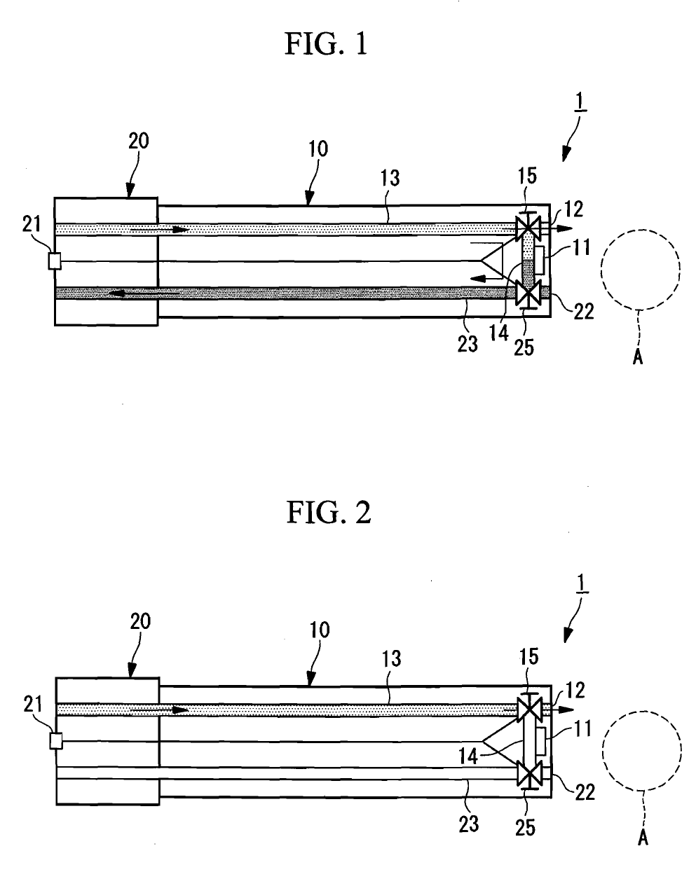

As shown in FIG. 1, an endoscope system 1 according to this embodiment is provided with, for example, an inserted portion 10, which is formed long and thin so as to be inserted inside a body cavity for acquiring an image of the inside of the body cavity; an operation portion 20, which actuates a fluid-supply-direction switching valve 15 and a suction-direction switching valve 25 (to be described later) provided in the inserted portion 10; and an endoscope control unit (not shown), which feeds cooling fluid to the inserted portion 10 and applies image processing, etc. to an image acquired by the inserted portion 10.

The inserted portion 10 is provided with photoelectric conversion devices 11 disposed at the distal end thereof; a fluid-feed channel 13 and a suction channel 23, which extend in the longitudinal direction along the entire length of the inserted portion 10 from a proximal end to the distal end, with an opening formed at the distal end thereof; the fluid-supply-direction switching valve (fluid-supply-direction switching portion) 15 provided at an intermediate position in the fluid-feed channel 13; the suction-direction switching valve (suction-direction switching portion) 25 provided at an intermediate position in the suction channel 23; and a radiator 14, which is disposed adjacent to the photoelectric conversion devices 11 and which is connected to the fluid-feed channel 13 and the suction channel 23 via the fluid-supply-direction switching valve 15 and suction-direction switching valve 25, respectively.

The photoelectric conversion devices 11 are, for example, a light-emitting diode used as a light source and a CCD used as an image acquisition device. Accordingly, an observation site A facing the distal end of the inserted portion 10 is irradiated with light from the light-emitting diode, and an image of the observation site A is acquired by the CCD.

The fluid-feed channel 13 has a fluid-feed port 12 opening at the distal end of the inserted portion 10 so as to flow the cooling fluid therethrough from the proximal end of the inserted portion 10 to the fluid-feed port 12. Accordingly, the cooling fluid is supplied to the observation site A facing the fluid-feed port 12 to wash the observation site A or an observation window (not shown) for the light-emitting diode and the CCD.

The radiator 14 is composed of metal material with a high heat conductivity and is configured so as to efficiently cool the photoelectric conversion devices 11 by ensuring a large contact area with the photoelectric conversion devices 11, the radiator 14 being, for example, a fluid-cooled heat exchanger disposed adjacent to the photoelectric conversion devices 11.

The fluid-supply-direction switching valve 15 is configured so as to switch a supply direction of the cooling fluid fed through the fluid-feed channel 13 from the proximal end of the inserted portion 10 to a fluid-feed port 12 side or to a radiator 14 side, on the basis of an instruction from an operating portion 20. Specific configurations of the fluid-supply-direction switching valve 15 include, for example, a three-way solenoid valve.

The suction channel 23 has a suction port 22 opening at the distal end of the inserted portion 10 so as to suck liquid or gas from the suction port 22 and discharge it to the proximal end of the inserted portion 10. In addition, the suction channel 23 is connected to the fluid-feed channel 13 via the radiator 14 so that it is possible to suck the cooling fluid fed to the radiator 14 and to discharge it to the proximal end of the inserted portion 10.

The suction-direction switching valve 25 is configured so as to switch a suction direction of the suction channel 23 to the suction port 22 side or to the radiator 14 side, on the basis of an instruction from the operating portion 20. Specific configurations of the suction-direction switching valve 25 include, for example, a three-way solenoid valve.

The operating portion 20 is provided with an operation switch 21 that outputs actuation instructions to the fluid-supply-direction switching valve 15 and the suction-direction switching valve 25, on the basis of a user operation.

The operation switch 21 is configured so as to make the fluid-supply-direction valve 15 and the suction-direction switching valve 25 actuate cooperatively. More specifically, when the fluid-supply-direction switching valve 15 is actuated so as to direct the fluid-feed direction of the fluid-feed channel 13 to the radiator 14 side, the suction-direction switching valve 25 is actuated so as to direct the suction-direction of the suction channel 23 to the radiator 14 side.

The operation of the endoscope system 1 configured as described above will be described below using FIGS. 1 to 3.

First, an observation in progress, that is, a case in which the cooling fluid is flowed through to the radiator 14, will be described.

As shown in FIG. 1, the operation switch 21 actuates the fluid-supply-direction switching valve 15 and the suction-direction switching valve 25 so as to set the fluid-feed direction of the fluid-feed channel 13 and the suction direction of the suction channel 23 to the radiator 14 side. In this case, the cooling fluid fed from the proximal end of the inserted portion 10 passing through the fluid-feed channel 13 is flowed through the radiator 14 and is then flowed through the suction channel 23 to be discharged to the proximal end of the inserted portion 10. Here, when flowing through the radiator 14, heat exchange between the photoelectric conversion devices 11 and the cooling fluid is carried out via the radiator 14, and thus, the heat generated from the photoelectric conversion devices 11 is transmitted to the cooling fluid and is discharged to the exterior. In this way, cooling of the photoelectric conversion devices 11 is carried out without supplying the cooling fluid to the observation site A.

Next, when feeding fluid to the observation site A, that is, a case in which the cooling fluid is supplied from the fluid-feed port 12, will be described.

As shown in FIG. 2, the operation switch 21 actuates the fluid-supply-direction switching valve 15 so as to set the fluid-feed direction of the fluid-feed channel 13 to the fluid-feed port 12 side. In this case, the cooling fluid fed from the proximal end of the inserted portion 10 passing through the fluid-feed channel 13 is supplied to the observation site A from the fluid-feed port 12, and thus, washing or cooling of the observation site A is carried out.

Next, the case of sucking from the observation site A, that is, the casein which liquid or gas is sucked from the suction port 22 will be described.

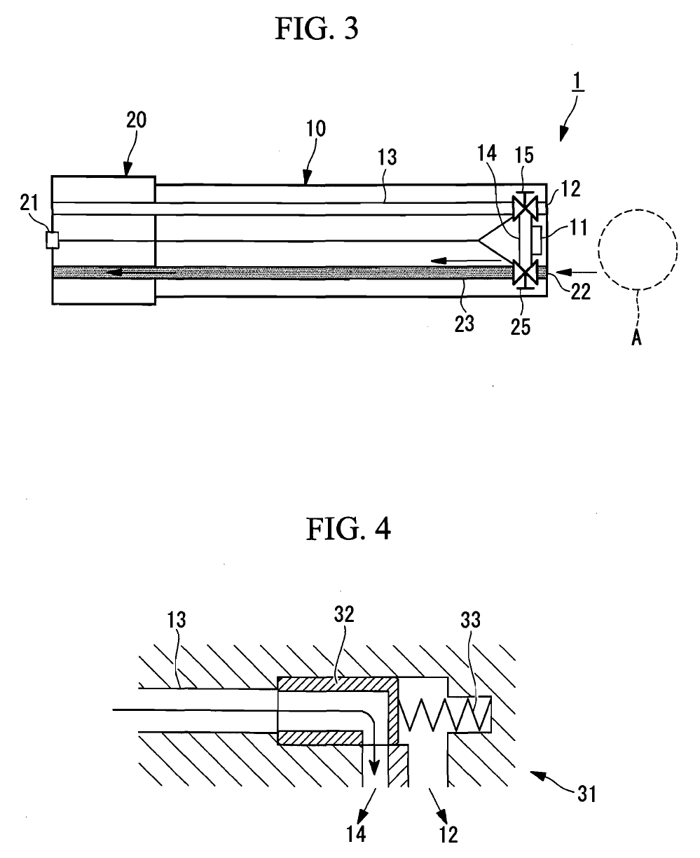

As shown in FIG. 3, the operation switch 21 actuates the suction-direction switching valve 25 so as to set the fluid-feed direction of the suction channel 23 to the suction port 22 side. In this case, liquid or gas in the vicinity of the suction port 22 is sucked from the suction port 22, is flowed through the suction channel 23, and is discharged to the proximal end of the inserted portion 10. Accordingly, fluid and the like that interferes with observation of the observation site A is removed.

As described above, in the endoscope system 1 according to this embodiment, it is possible to carry out washing of the observation site A facing the fluid-feed port 12 or the observation window (not shown) for the light-emitting diode and the CCD, by supplying the cooling fluid via the fluid-feed channel 13 while the inserted portion 10 is inserted in the body cavity. On the other hand, the photoelectric conversion devices 11 mounted at the distal end of the inserted portion 10 can be cooled by switching the fluid-supply direction with the fluid-supply-direction switching valve 15 to supply the cooling fluid to the radiator 14 side. Accordingly, the photoelectric conversion devices 11 can be cooled using the fluid-feed channel 13 which feeds the cooling fluid to the fluid-feed port 12 side, and because a separate fluid-feed channel for cooling the photoelectric conversion devices 11 need not be provided, it is possible to prevent an increase in the outer diameter of the inserted portion 10.

In addition, by providing the suction channel 23 for sucking liquid or gas in the vicinity of the suction port 22, fluid and the like that interferes with observation near the distal end of the inserted portion 10 can be removed, thus simplifying the observation procedure.

Furthermore, by switching the suction direction of the suction channel 23 with the suction-direction switching valve 25, fluid fed for washing or cooling of the observation site A facing the fluid-feed port 12 and fluid fed for cooling the radiator 14 can be sucked by the suction channel 23. Therefore, because a separate return channel for returning fluid used to cool the radiator 14 need not be provided, it is possible to reduce the outer diameter of the inserted portion 10.

Note that in the above-described endoscope system 1, feeding of fluid to the observation site A shown in FIG. 2 and suction from the observation site A shown in FIG. 3 may be performed simultaneously. In this case, when the fluid-supply-direction switching valve 15 is actuated so as to set the fluid-feed direction of the fluid-feed channel 13 to the fluid-feed port 12 side, the suction-direction switching valve 25 is actuated so as to set the suction direction of the suction channel 23 to the suction port 22 side. Accordingly, it is possible to enhance washability of the observation site A.

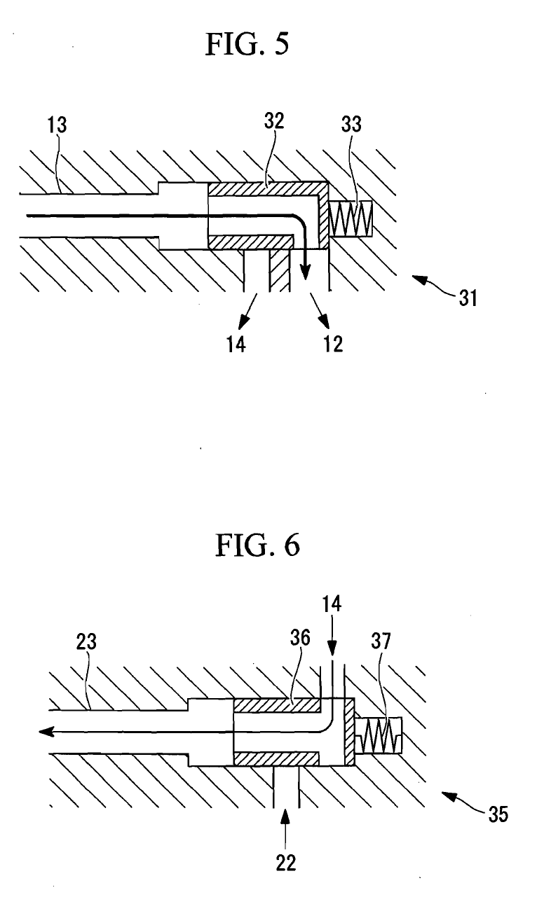

In addition, as shown in FIGS. 4 and 5, a fluid-supply direction switching valve (fluid-supply-direction switching portion) 31, which switches the supply direction of the cooling fluid by using pressure in the fluid-feed channel 13, may be employed as a first modification of this embodiment. Here, FIG. 4 shows the state of the fluid-supply-direction switching valve 31 during observation (low fluid pressure, low flow rate), and FIG. 5 shows the state of the fluid-supply-direction switching valve 31 during fluid-feeding (high fluid pressure, high flow rate).

This fluid-supply-direction switching valve 31 includes a valve piece 32, which is disposed so as to be movable in the direction of cooling fluid flow between two positions that alternately communicate the fluid-feed channel 13 with a flow path to the fluid-feed port 12 side and a flow path to the radiator 14 side, and a spring 33 disposed between the valve piece 32 and an inner wall of the fluid-feed channel 13 to bias the valve piece 32 toward the upstream side.

In the fluid-supply-direction switching valve 31 having the above-described configuration, when a pressure is applied to the cooling fluid in the fluid-feed channel 13, the valve piece 32 is pressed toward the downstream side by this pressure, and the supply direction of the cooling fluid being flowed through the fluid-feed channel 13 is switched to the fluid-feed port 12 side or to the radiator 14 side.

As described above, with the endoscope system according to the first modification, the fluid-supply-direction switching valve 31 is actuated by applying pressure to the cooling fluid in the fluid-feed channel 13, and thus, it is possible to feed fluid by selecting the fluid-feed port 12 side or the radiator 14 side. Accordingly, the fluid-supply-direction switching valve 31 can be of a simple configuration in which a spring, etc. is used, instead of a configuration with a solenoid valve, etc., which requires electric power. In addition, because a power cable for actuating a solenoid valve, etc. need not be provided in the inserted portion 10, it is possible to prevent an increase in the outer diameter of the inserted portion 10.

Furthermore, with the above-described fluid-supply-direction switching valve 31, when the pressure in the fluid-feed channel 13 is below a predetermined value, the supply direction of the cooling fluid may be switched to the radiator 14 side, and when the pressure in the fluid-feed channel 13 is at or above the predetermined value, the supply direction of the cooling fluid may be switched to the fluid-feed port 12 side.

In this way, as shown in FIG. 4, the cooling fluid supplied to the radiator 14 side is set at a low pressure and a low flow rate, and it is possible to reduce the fluid pressure exerted on the radiator 14. In addition, as shown in FIG. 5, the cooling fluid supplied to the fluid-feed port 12 side is set at a high pressure and a high flow rate, and it is possible to improve washability of the observation site A facing the fluid-feed port 12.

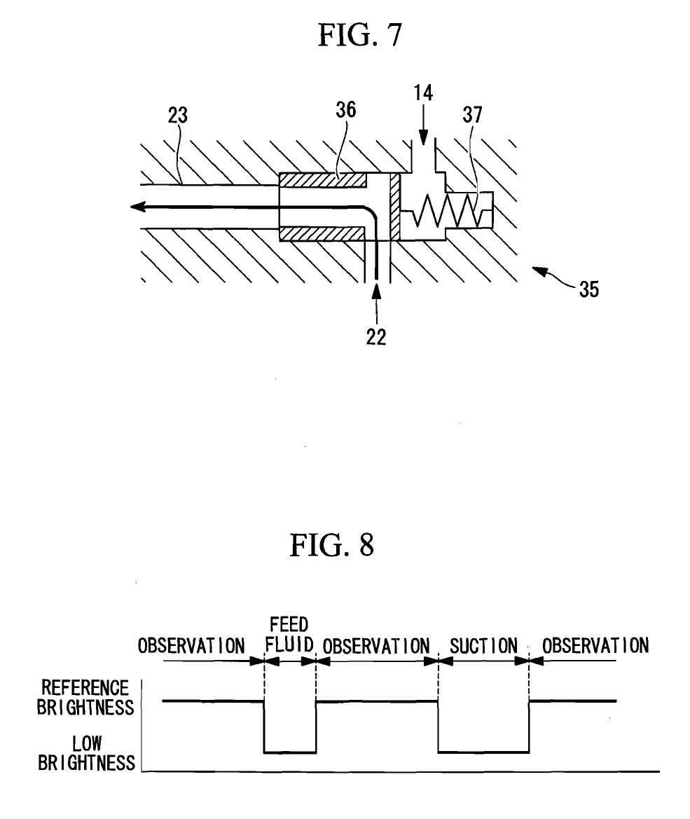

In addition, as shown in FIGS. 6 and 7, a suction-direction switching valve (suction-direction switching portion) 35, which switches the suction direction of the cooling fluid by using pressure in the suction channel 23, may be employed as a second modification of this embodiment. Here, FIG. 6 shows the state of the suction-direction switching valve 35 during observation (no suction pressure), and FIG. 7 shows the state of the suction-direction switching valve 35 during suction (with suction pressure).

This suction-direction switching valve 35 is provided with a valve piece 36, which is disposed so as to be movable in the suction direction of fluid, etc. between two positions that alternately communicate the suction channel 23 with a flow path to the suction port 22 side and a flow path to the radiator 14 side, and a spring 37 disposed between the valve piece 36 and an inner wall of the suction channel 23 to pull the valve piece 36 toward the upstream side.

In the suction-direction switching valve 35 having the above-described configuration, when a suction pressure is applied in the suction channel 23, the valve piece 36 is pulled toward the downstream side, and the suction direction of the suction channel 23 is switched to the suction port 22 side or the radiator 14 side.

As described above, with the endoscope system according to the second modification, the suction-direction switching valve 35 is actuated by changing the pressure in the suction channel 23, and thus, it is possible to suck by selecting the suction port 22 side or the radiator 14 side. Accordingly, the suction-direction switching valve 35 can be of a simple mechanism, in which a spring, etc. is used, instead of a mechanism with a solenoid valve, etc., which requires electric power. In addition, a power cable for actuating a solenoid valve, etc. need not be provided in the inserted portion 10, and it is possible to prevent an increase in the outer diameter of the inserted portion 10.

Furthermore, as shown in FIG. 8, a low-heat-generation mode setting portion (not shown), which sets the photoelectric conversion devices 11 to low-heat-generation modes, when the fluid-supply-direction switching valve 15 or 31 switches the supply direction of the cooling fluid to the fluid-feed port 12 side, may be additionally provided as a third modification of this embodiment.

In this way, when the fluid-supply-direction switching valve 15 or 31 sets the supply direction of the cooling fluid to the fluid-feed port 12 side, that is, when the photoelectric conversion devices 11 are not being cooled, the low-heat-generation mode setting portion sets the photoelectric conversion devices 11 to the low-heat-generation modes, and thus, the amount of heat generated can be reduced. Here, for example, a light-emitting diode, a CCD, and the like are used as the photoelectric conversion devices 11, and examples of low-heat-generation modes for these include lowering the light emission level of a light-emitting diode and lowering the operating clock speed of a CCD.

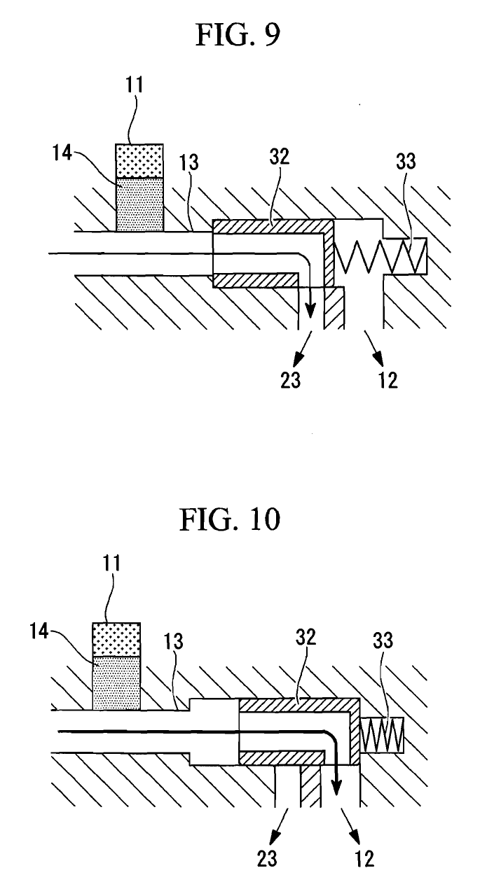

In addition, as shown in FIGS. 9 and 10, as a fourth modification of this embodiment, the photoelectric conversion devices 11 and the radiator 14 may be provided upstream of a flow-path branching point of the fluid-feed channel 13 such that the cooling fluid is flowed through the radiator 14 in both cases when the cooling fluid is fed to the suction channel 23 side and when it is fed to the fluid-feed port 12 side. Here, FIG. 9 shows the state of the valve piece 32 during observation (low fluid pressure, low flow rate), and FIG. 10 shows the state of the valve piece 32 when fluid is being fed (high fluid pressure, high flow rate).

In this way, the photoelectric conversion devices 11 can always be cooled by the radiator 14 regardless of the feeding direction of the cooling fluid.

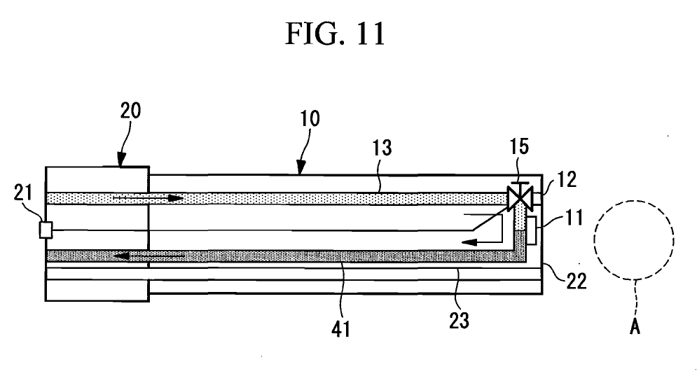

In addition, as a fifth modification of this embodiment, as shown in FIG. 11, a return channel 41 that returns the cooling fluid used to cool the photoelectric conversion devices 11 to the proximal end of the inserted portion 10 may be provided separately from the suction channel 23.

In this way, obstacles such as a valve, etc. in the suction channel 23 can be eliminated, and solid matter such as undigested food, etc. can be prevented from blocking the suction channel 23.

### Second Embodiment

Next, a second embodiment of the present invention will be described below.

An endoscope system according to this embodiment differs from the first embodiment in that cooling fluid is allowed to seep out to a surface of a radiator, and photoelectric conversion devices are cooled by the heat of vaporization of this cooling fluid. In the following description of an endoscope system of this embodiment, commonalities with the first embodiment will be omitted and differences will be mainly described.

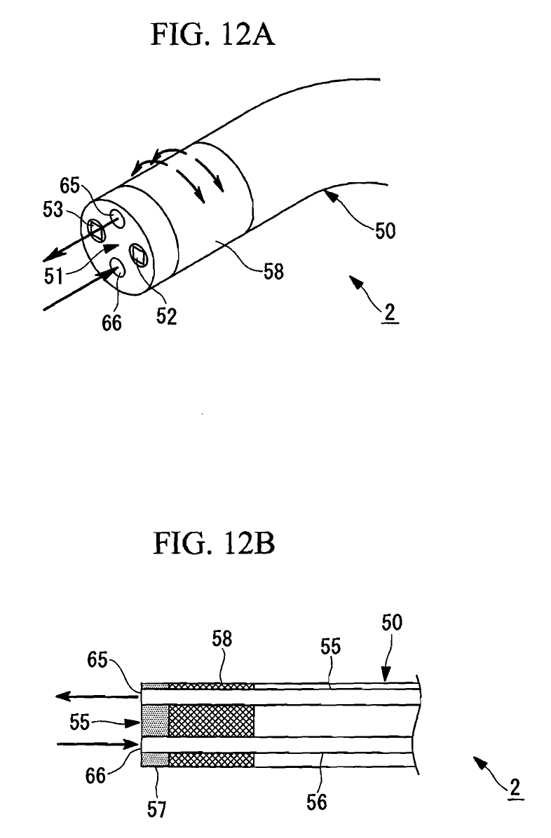

As shown in FIGS. 12A and 12B, an endoscope system 2 according to this embodiment is provided with, for example, an inserted portion 50, which is inserted inside a body cavity, and an endoscope control unit (not shown), which feeds cooling fluid to the inserted portion 50 and applies image processing, etc. to an image acquired by the inserted portion 50.

The inserted portion 50 is provided with a securing portion 57 disposed at a distal end thereof; a light source 52 and a CCD 53 (referred to hereinafter as “photoelectric conversion devices 51”) that are secured on the securing portion 57; a fluid-feed channel 55 and a suction channel 56, which extend in the longitudinal direction along the entire length of the inserted portion 50 from a proximal end to a distal end, with an opening formed at the distal end thereof; and a radiator 58, which is disposed at the distal end of the inserted portion 50 adjacent to the photoelectric conversion devices 51.

The fluid-feed channel 55 has a fluid-feed port 65 opening at the distal end of the inserted portion 50 so as to flow the cooling fluid therethrough from the proximal end of the inserted portion 50 to the fluid-feed port 65. Accordingly, the cooling fluid is supplied to an observation site A facing the fluid-feed port 65 to wash the observation site A or an observation window (not shown) for the light-emitting diode and the CCD.

The suction channel 56 has a suction port 66 opening at the distal end of the inserted portion 50 so as to suck liquid or gas from the suction port 66 and discharge it to the proximal end of the inserted portion 50.

The radiator 58 is constituted of a porous material having numerous pores, from which the cooling fluid fed from the fluid-feed channel 55 in communication therewith seeps out, wherein the pores are provided on an outer surface at the distal end of the inserted portion 50. By configuring the radiator 58 in this way, the cooling fluid in the fluid-feed channel 55 seeps out to the surface of the radiator 58 though the numerous pores, and heat dissipation is carried out because the seeped out cooling fluid takes away the heat of vaporization from the radiator 58 when vaporizing. Note that, as a porous material constituting the radiator 58, for example, a sintered metal is suitable.

The operation of the endoscope system 2 having the above-described configuration will be described below.

When the inserted portion 50 is inserted inside a body cavity and observation in the body cavity begins, the cooling fluid is fed to the fluid-feed channel 55 by the endoscope control unit.

The cooling fluid that is fed from the proximal end of the inserted portion 50 through the fluid-feed channel 55 is supplied to the observation site A from the fluid-feed port 65, and washing or cooling of the observation site A is carried out. In addition, part of the cooling fluid flowing in the fluid-feed channel 55 therethrough seeps out to the surface of the radiator 58 through the numerous pores therein. Heat dissipation is carried out when the fluid that seeps out in this way vaporizes because the heat of vaporization is taken away from the radiator 58.

As described above, in the endoscope system 2 according to this embodiment, the cooling fluid in the fluid-feed channel 55 seeps out to the surface of the radiator 58 through the numerous pores therein, and heat dissipation is carried out when the fluid that seeps out vaporizes because the heat of vaporization is taken away from the radiator 58; therefore, the photoelectric conversion devices 51 can be cooled.

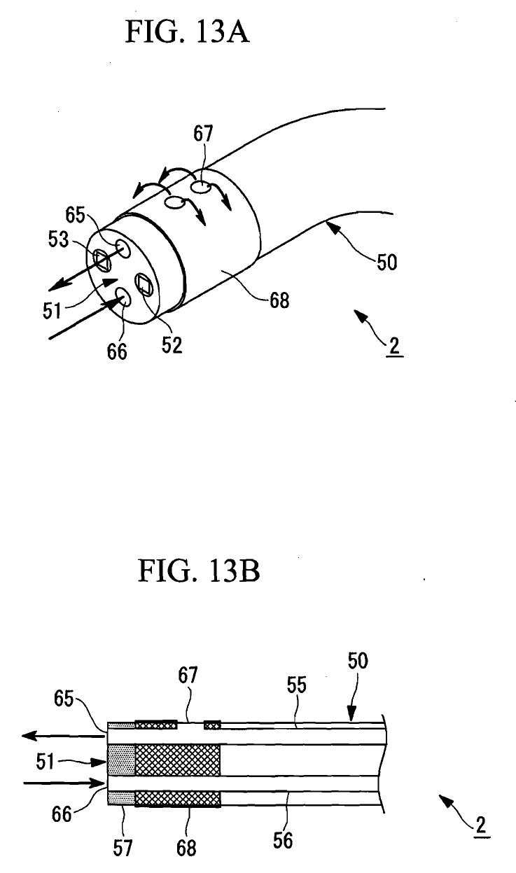

Note that, as a modification of this embodiment, as shown in FIGS. 13A and 13B, instead of forming the radiator of a porous material, a hydrophilic layer 68 may be formed on a heat dissipating surface of the radiator, by providing an opening portion 67, which supplies the cooling fluid fed in communication with the fluid-feed channel 55, at the distal end of the inserted portion 50 on an outer surface thereof.

In this way, the cooling fluid supplied from the opening portion 67 can spread on the hydrophilic layer 68 formed on the heat dissipating surface, and heat dissipation can be efficiently carried out. Note that, as the hydrophilic layer 68, for example, a photocatalyst such as titanium oxide, etc. is suitable.

### Third Embodiment

Next, a third embodiment of the present invention will be described below.

An endoscope system according to this embodiment differs from the second embodiment in that detectors that detect conditions in a body cavity are provided at a distal end of an inserted portion to carry out heat dissipation from a radiator on the basis of detected values. In the following description of an endoscope system of this embodiment, commonalities with the above-described embodiments will be omitted and differences will be mainly described.

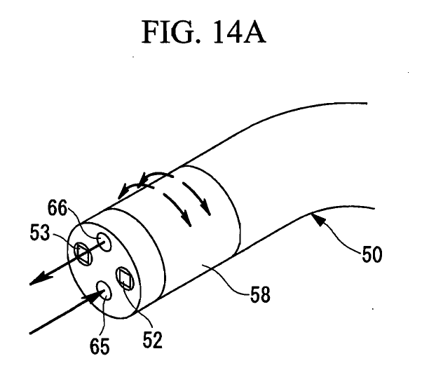

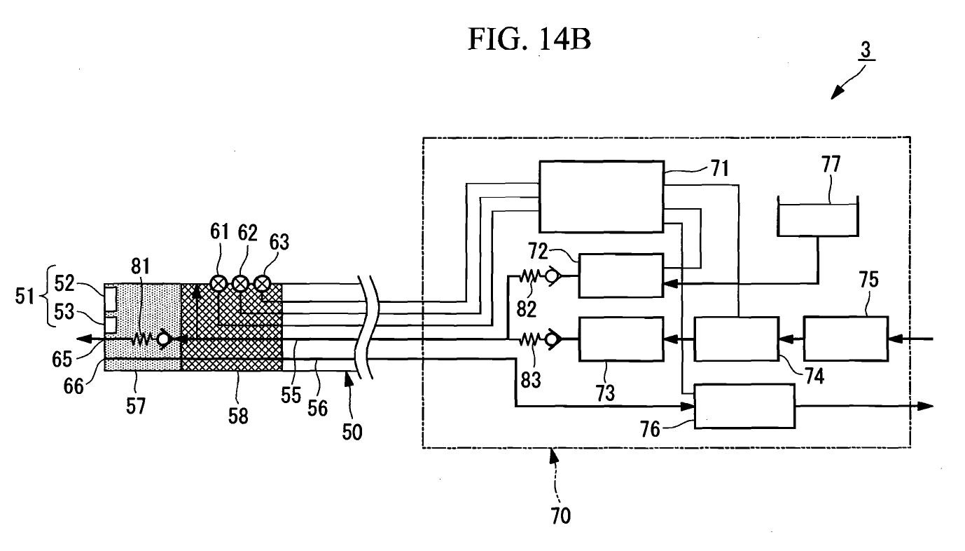

As shown in FIGS. 14A and 14B, an endoscope system 3 according to this embodiment is provided with, for example, an inserted portion 50 that is inserted in a body cavity, and an endoscope control unit 70, which feeds cooling fluid to the inserted portion 50 and applies image processing, etc. to an image acquired by the inserted portion 50.

The inserted portion 50 is provided with a securing portion 57 disposed at the distal end thereof; photoelectric conversion devices 51 that are secured to the securing portion 57; a radiator 58 disposed adjacent to the photoelectric conversion devices 51; a fluid-feed channel 55 and a suction channel 56 provided in the longitudinal direction along the entire length of the inserted portion 50; a fluid-supply valve 81 provided further downstream from the radiator 58 of the fluid-feed channel 55; and a temperature detector 61 that detects the temperature of the radiator 58, a humidity detector 62 that detects the humidity around the radiator 58, and a pressure detector 63 that detects the pressure around the radiator 58, which are provided adjacent to the radiator 58.

The endoscope control unit 70 is provided with a tank 77 that retains cooling fluid; a fluid-feeding pump 72 that pumps the cooling fluid from the tank 77 to the fluid-feed channel 55; a fluid-feed valve 82 that is provided on a secondary side of the fluid-feeding pump 72; an air-feeding pump (air-feeding portion) 74 that takes in air from the exterior of the endoscope control unit 70 and pumps it to the fluid-feed channel 55; an air filter 75 that is installed on a primary side of the air-feeding pump 74; a dryer 73 that is installed on the primary side of the air-feeding pump 74 to dry the air; an air-feed valve 83 that is provided on a secondary side of the dryer 73; a discharge/exhaust pump 76 that sucks the cooling fluid and air from the suction channel 56; and a controller (a fluid-feeding level adjusting portion and a suction level adjusting portion) 71 that controls the fluid-feeding pump 72, the air-feeding pump 74, and the discharge/exhaust pump 76.

The fluid-supply valve 81, fluid-feed valve 82, and the air-feed valve 83 are respectively configured so as to close a flow path under a condition where pressure lower than a set value is applied, and to open the flow path to allow the cooling fluid or the air to flow therethrough under a condition where pressure equal to or greater than the set value is applied. Here, the set value for the fluid-supply valve 81 is set higher than the set values for the fluid-feed valve 82 and the air-feed valve 83 so as to achieve a state in which the fluid-supply valve 81 is closed whereas the fluid-feed valve 82 and the air-feed valve 83 are open, by making a delivery pressure of the fluid-feeding pump 72 or the air-feeding pump 72 fall within a predetermined range. In this way, it is possible to supply the cooling fluid or air to the radiator 58 to carry out heat dissipation of the radiator 58 without feeding fluid or feeding air to an observation site A from a fluid-feed port 65.

The controller 71 is configured so as to adjust the fluid-feeding level of the fluid-feeding pump 72 in accordance with the temperature detected by the temperature detector 61. The controller 71 is also configured so as to adjust the air-feeding level of the air-feeding pump 74 in accordance with the humidity detected by the humidity detector 62. The controller 71 is further configured so as to adjust the suction level of the discharge/exhaust pump 76 in accordance with the pressure detected by the pressure detector 63.

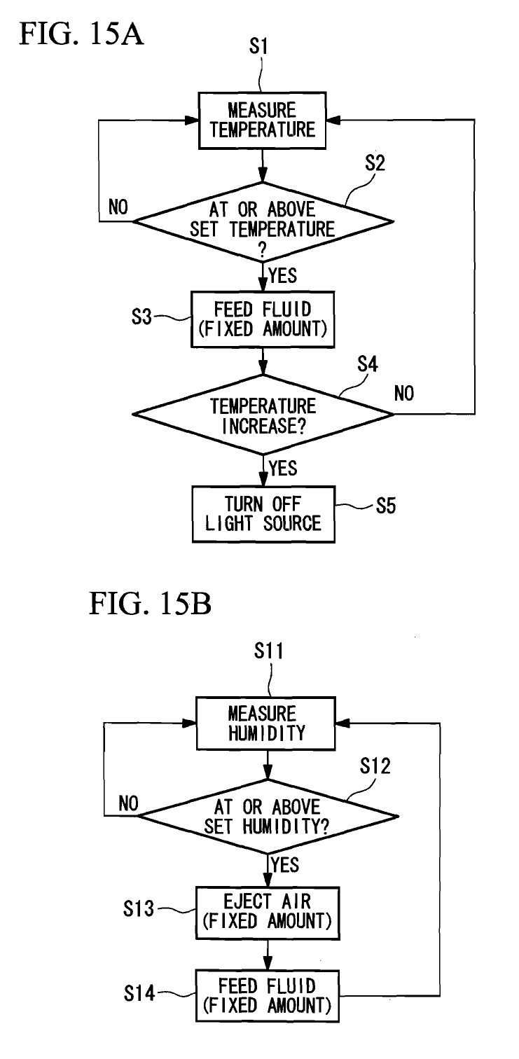

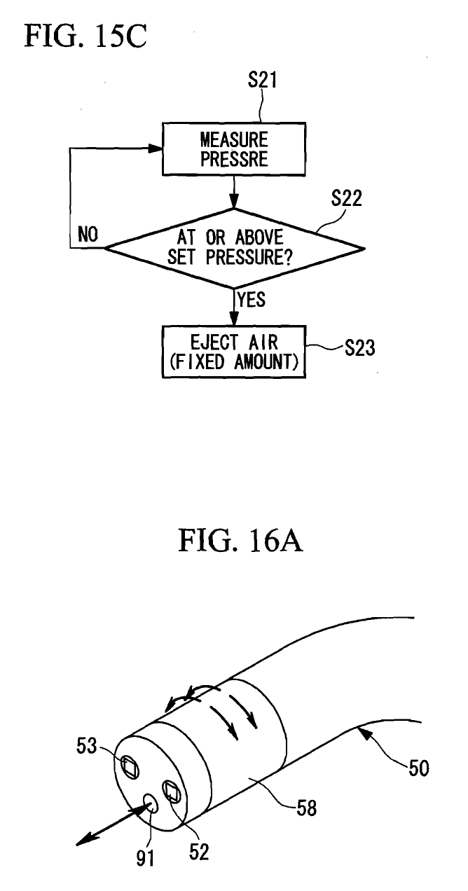

Specific control by the above-described controller 71 will be described below based on flow charts shown in FIG. 15.

As shown in FIG. 15A, the temperature of the radiator 58 is measured by the temperature detector 61 (S1), and when the measurement result is at or above a set temperature (S2), a fixed amount of the cooling fluid is fed to the radiator 58 by the fluid-feeding pump 72 (S3). Then, the temperature change of the radiator 58 is determined (S4); when the temperature of the radiator 58 decreases, the temperature measurement of the radiator 58 is continued (S1); and when the temperature of the radiator 58 increases, it is determined that an abnormality such as a blockage in the fluid-feed channel, etc. has occurred, and a light source 52 is turned off (S5).

In addition, as shown in FIG. 15B, the humidity around the radiator 58 is measured by the humidity detector 62 (S11); when the measurement result is at or above a set humidity (S12), a fixed amount of air, etc. is sucked by the discharge/exhaust pump 76 to be ejected to the exterior of the inserted portion 50 (S13), and a fixed amount of low-temperature air is fed to the radiator 58 by the air-feeding pump 74 (S14).

Furthermore, as shown in FIG. 15C, the pressure around the radiator 58 is measured by the pressure detector 63 (S21); when the measurement result is at or above a set pressure (S22), a fixed amount of air, etc. is sucked by the discharge/exhaust pump 76 to be ejected to the exterior of the inserted portion 50 (S23)

The operation of the endoscope system 3 having the above-described configuration will be described below.

When the inserted portion 50 is inserted inside a body cavity and observation in the body cavity begins, the cooling fluid is fed to the fluid-feed channel 55 by the fluid-feeding pump 72.

The cooling fluid that is fed from the proximal end of the inserted portion 50 through the fluid-feed channel 55 is supplied to an observation site A from the fluid-feed port 65, and washing or cooling of the observation site A is carried out. In addition, part of the cooling fluid flowing in the fluid-feed channel 55 therethrough seeps out to a surface of the radiator 58 through numerous pores therein. Heat dissipation is carried out when the fluid that seeps out in this way vaporizes because the heat of vaporization is taken away from the radiator 58.

When most of the cooling fluid that has seeped out onto the radiator 58 evaporates, the temperature of the radiator 58 increases due to a decrease in the heat dissipation level. The temperature of the radiator 58 is detected by the temperature detector 61, and when the detected temperature reaches or exceeds a preset temperature, the controller 71 turns on the fluid-feeding pump 72 for a fixed duration to feed a fixed amount of the cooling fluid to the fluid-feed channel 55. Part of the cooling fluid fed in this way seeps out to the surface of the radiator 58 and evaporates, and, during this process, heat dissipation of the radiator 58 is carried out again due to an endothermic effect. The above processes are repeated until the temperature detected by the temperature detector 61 decreases below the preset temperature.

On the other hand, when a fixed amount or greater of the cooling fluid vaporizes, the humidity in the body cavity increases, and the evaporation level of the cooling fluid at the radiator 58 decreases. The humidity in the body cavity is detected by the humidity detector 62, and when the detected humidity reaches or exceeds a preset humidity, the air-feeding pump 74 is turned on for a fixed duration to feed a fixed amount of air to the radiator 58. Here, dry air is fed into the body cavity because the air fed by the air-feeding pump 74 is dried by the dryer 73. In this way, the humidity in the body cavity is deceased, facilitating vaporization of the cooling fluid at the radiator.

Here, a patient may experience a volume expansion due to vaporization of the cooling fluid or an increase in pressure in the body cavity due to feeding of air by the air-feeding pump 74. The pressure in the body cavity is detected by the pressure detector 63, and when the detected pressure reaches or exceeds a preset pressure, the discharge/exhaust pump 76 is turned on for a fixed duration to suck a fixed amount of air, etc. to be ejected to the exterior of the inserted portion 50. In this way, the pressure in the body cavity is decreased, and the pressure in the body cavity is maintained within a fixed range.

As described above, with the endoscope system 3 according to this embodiment, by providing the temperature detector 61 that detects the temperature of the radiator 58 and the fluid-feeding pump 72 that adjusts the fluid-feeding level to the radiator 58 in accordance with the temperature detected by the temperature detector 61, it is possible to perform temperature management of the radiator 58 with the temperature detector 61 and to prevent the radiator 58 from overheating.

In addition, by providing the air-feeding pump 72 that feeds air to the fluid-feed channel 55, it is possible to feed air to the radiator 58 via the fluid-feed channel 55 with the air-feeding pump 72 and to facilitate vaporization at the radiator 58. Note that, this embodiment has been described such that air fed by the air-feeding pump 72 as being flowed in the fluid-feed channel 55 therethrough; however, an air-feed channel that opens near the radiator 58 may be provided in the inserted portion 50, separately from the fluid-feed channel 55.

Furthermore, by providing the humidity detector 62 that detects the humidity around the radiator 58 and by having the air-feeding pump 72 feed air when the humidity detected by the humidity detector 62 reaches or exceeds the predetermined value, management of the humidity around the radiator 58 is performed by the humidity detector 62, and when detected humidity is at or above the predetermined value, the air-feeding pump 72 feeds air to the radiator 58; therefore it is possible to facilitate vaporization at the radiator 58.

In addition, by providing the pressure detector 63 that detects the pressure around a suction port 66 and the discharge/exhaust pump 76 that adjusts the suction level from the suction port 66 in accordance with the pressure detected by the pressure detector 63, it is possible to suck, with the suction channel 56, the cooling fluid that is heated upon being used to cool the radiator 58, and to maintain the pressure in the body cavity at an appropriate value.

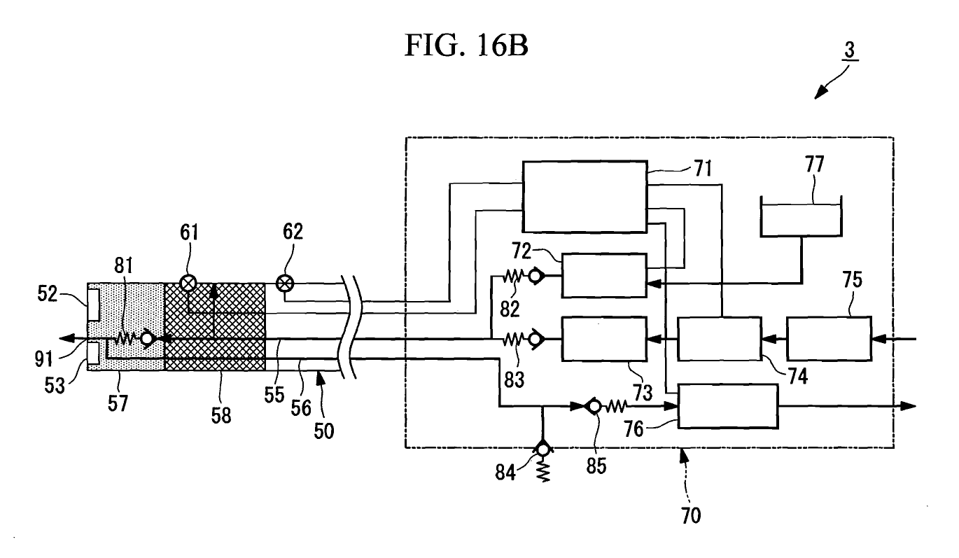

Note that, as shown in FIGS. 16A and 16B, instead of providing the pressure detector 63, a pressure relief valve 84 that opens a flow path at a predetermined pressure may be provided on the primary side of the discharge/exhaust pump 76 as a modification of this embodiment.

In this way, it is possible to release the pressure in the body cavity via the pressure-open valve 84 when a predetermined pressure is reached or exceeded in the body cavity. In addition, the cooling fluid may be supplied and sucked from the fluid-feed-suction port 91 by providing a discharge valve 85 on the primary side of the discharge/exhaust pump 76 and by connecting the fluid-feed channel 55 and the suction channel 56.

Embodiments of the present invention have been described above with reference to the drawings; however, specific configurations are not limited to these embodiments, and design alterations that do not depart from the gist of the present invention are also encompassed.

For example, the endoscope system 1 according to the first embodiment may be provided with the temperature detector 61 according to the third embodiment and may be configured to feed the cooling fluid in accordance with temperature detected by the temperature detector 61.

1. An endoscope system comprising:
a long, thin inserted portion;
a photoelectric conversion device that is mounted at a distal end of the inserted portion;
a fluid-feed channel that is provided in the inserted portion and that has a fluid-feed port which opens at the distal end of the inserted portion;
a radiator that is connected to an intermediate position of the fluid-feed channel and that is provided in a manner enabling heat exchange with the photoelectric conversion device; and
a fluid-supply-direction switching portion that switches a supply direction of cooling fluid fed by the fluid-feed channel to the fluid-feed port side or to the radiator side.

2. An endoscope system according to claim 1, wherein the fluid-supply-direction switching portion switches the supply direction using pressure in the fluid-feed channel.

3. An endoscope system according to claim 2, wherein, when the pressure in the fluid-feed channel is less than a predetermined value, the fluid-supply-direction switching portion switches the supply direction of the cooling fluid to the radiator side and, when the pressure in the fluid-feed channel is at or above the predetermined value, switches the supply direction of the cooling fluid to the fluid-feed port side.

4. An endoscope system according to claims 1 further comprising a low-heat-generation-mode setting portion that sets the photoelectric conversion device to low-heat-generation modes, when the fluid-supply-direction switching portion switches the supply direction to the fluid-feed port side.

5. An endoscope system according to claims 1 further comprising a suction channel that is provided in the inserted portion, that has a suction port provided at the distal end of the inserted portion, and that sucks liquid or gas from the suction port.

6. An endoscope system according to claim 5, in which the fluid-feed channel and the suction channel are connected via the radiator, further comprising:
a suction-direction switching portion that switches the suction direction of the suction channel to the suction port side or to the radiator side,
wherein the suction-direction switching portion switches the suction direction of the suction channel to the radiator side when the fluid-feeding direction of the fluid-feed channel is set to the radiator side.

7. An endoscope system according to claim 6, wherein the suction-direction switching portion switches the suction direction using pressure in the suction channel.

8. An endoscope system comprising:
a long, thin inserted portion;
a photoelectric conversion device that is mounted at a distal end of the inserted portion;
a fluid-feed channel that is provided in the inserted portion, that has a fluid-feed port which opens at the distal end of the inserted portion, and that feeds cooling fluid to the fluid-feed port; and
a radiator that is disposed adjacent to the photoelectric conversion device, that opens to an outer surface of the inserted portion at the distal end thereof, and from which the cooling fluid fed from the fluid-feed channel in communication therewith seeps out.

9. An endoscope system according to claim 8, wherein the radiator is constituted of a porous material having numerous pores.

10. An endoscope system according to claim 8, wherein the radiator has a hydrophilic layer on a heat dissipation surface thereof.

11. An endoscope system according to claims 8 further comprising:
a temperature detector that detects the temperature of the radiator; and
a fluid-feeding level adjusting portion that adjusts a fluid-feeding level to the radiator in accordance with the temperature detected by the temperature detector.

12. An endoscope system according to claims 8 further comprising:
an air-feed channel that is provided in the inserted portion and that opens near the radiator; and
an air-feeding portion that feeds air to the air-feed channel.

13. An endoscope system according to claim 12 further comprising a humidity detector that detects the humidity around the radiator, wherein
the air-feeding portion feeds air when the humidity detected by the humidity detector is at or above a predetermined value.

14. An endoscope system according to claims 8 further comprising:
a suction channel that is provided in the inserted portion, that has a suction port which opens at the distal end of the inserted portion, and that sucks liquid or gas near the suction port;
a pressure detector that detects the pressure around the suction port; and
a suction level adjusting portion that adjusts a suction level from the suction port in accordance with the pressure detected by the pressure detector.
